(19) United States
(12) Patent Application Publication
(10) Pub. No.: US 2013/0158588 A1
(43) Pub. Date: Jun. 20, 2013

(54) Abdominoplasty Umbilicus Finder Device and Method

(71) Applicant: Innospan Enterprises, Inc., Draper, UT (US)

(72) Inventor: Steven Warnock, Salt Lake City, UT (US)

(73) Assignee: INNOSPAN ENTERPRISES, INC., Draper, UT (US)

(21) Appl. No.: 13/712,846

(22) Filed: Dec. 12, 2012

Related U.S. Application Data
(60) Provisional application No. 61/570,728, filed on Dec. 14, 2011.

(51) Int. Cl. A61B 17/02

(52) U.S. Cl. CPC A61B 17/02
USPC 606/190

(57) ABSTRACT

A surgical device and method for locating the umbilicus during abdominoplasty surgery may include a device having a body and one or more projections. The body may be generally oval-shaped and fit around the umbilical pedicle. The device may be tacked into place with tacking sutures, by use of anchoring hooks on the body of the device, or otherwise held in place to assist in locating the proper location for a new umbilicus orifice during surgery. The one or more projections may be easily felt under the subcutaneous fat after all the layers of fat and skin have been closed. The method allows simple and accurate determination of the umbilicus following abdominoplasty to ensure proper positioning of the new orifice for the umbilicus.

### BACKGROUND OF THE INVENTION

1. The Field of the Invention

The present invention relates to a surgical device and method for use in locating the umbilicus during an abdominoplasty procedure. More specifically, the present invention relates to a device which may be placed around the umbilicus and can be felt through the layers of skin and fat to precisely locate the umbilicus.

2. State of the Art

Abdominoplasty surgery has been known for over 100 years, and has grown increasingly popular in recent years. Abdominoplasty methods vary, and can even be accomplished by endoscopic means in some cases. However, a “complete” abdominoplasty procedure involves the substantially same steps. First, an incision is made from hip to hip in the lower abdomen. A second incision is then made around the umbilicus, which is attached to the abdominal fascia tissue and is not repositioned. The skin and fat of the abdominal wall are then undermined and elevated as a flap, leaving the umbilicus still attached to the fascia below. After lifting the abdominal flap, the surgeon then tightens the abdominal wall muscles using suture, stretches the flap downward, removing the excess skin, and sutures the flap into place (preferably below the panty line for cosmetic purposes). Once the flap is sutured in place, the surgeon must locate the umbilicus, cut a new orifice, bring the umbilicus back through the new orifice, and suture it into place. Abdominoplasty, though routinely performed for aesthetic purposes, has several possible complications.

One of the more difficult aspects of successful abdominoplasty surgery relates to locating the umbilicus after the abdominal flap has been stretched and sutured into place. It is difficult to locate the exact location of the umbilicus under the subcutaneous fat layer. Proper location is critical; cutting the new orifice even 0.63 centimeters (¼ of an inch) in the wrong direction can have significant aesthetic implications. The new belly button will not have the appearance of a normal belly button (which is critical because an abdominoplasty is usually performed for the sole purpose of aesthetics) and further surgical revision must be performed. Thus, it is important to correctly locate the umbilicus and position the new orifice accurately at the outset.

Furthermore, the orifice for the umbilicus cannot be determined prior to at least partially closing the abdominal wall because the final position of the umbilicus shifts as the surgeon closes the various layers. Thus, the final position of the new orifice for the umbilicus is difficult to accurately determine until every layer has been closed.

According to the known prior art, surgeons do not have a device to adequately assist them in locating the umbilicus. Locating the umbilicus under the subcutaneous fat later varies greatly by the skill of the surgeon. One device, see U.S. Pat. No. 7,160,254, used for protecting the umbilicus and umbilical pedicle during abdominoplasty, could possibly be used later in the surgery for locating the orifice, but is not simple to use in practice and does not include projections that would be easy to feel under the enclosed skin.

Thus, there is a need for an improved device and method for locating the umbilicus following relocation of abdominal skin to complete abdominoplasty surgery, and to assure proper positioning of the new umbilicus orifice. It is further desired that such a device and method be simple and easy to deploy in surgery, eliminating or greatly reducing the need for surgeons with special skills or experience in umbilical placement.

### SUMMARY OF THE INVENTION

The present invention relates to an umbilicus finder device and methods for using the same. According to one aspect of the invention, the device is a generally oval-shaped body with at least one projection extending outwardly, i.e., 90 degrees out of the place of the body. The projection may be felt through the layers of skin and fat for simple tactile location.

One aspect of the invention relates to using two projections on the generally oval-shaped body. In yet another aspect, four projections may be used, and the four projections may be evenly spaced around the body of the device, such as at the 0, 90, 180, and 270 degree positions around the oval body.

According to another aspect of the invention, the device relates to a generally oval-shaped body with a short radius and a long radius. By way of example, the short radius may be between approximately 10-30 millimeters, and the long radius may be between approximately 30 and 50 millimeters.

According to another aspect of the invention, the device may be provided with anchoring hooks extending downwardly, in an opposite direction of the projections. The anchoring hooks may provide a simple means for attaching the device to the fascia.

According to another aspect of the invention, a method is provided for locating an umbilicus during abdominoplasty surgery. The method comprises attaching an umbilicus finder device to the fascia of the abdominal wall adjacent to the umbilicus, locating the umbilicus by feeling the umbilicus finder device through skin covering the abdomen, and forming a new umbilicus orifice.

According to another aspect of the invention, the method may include attaching the umbilicus finder device by use of anchoring hooks provided on the device, or by use of tacking sutures. The method may also include removing the umbilicus finder device through the new umbilicus orifice.

These and other aspects of the present invention may be realized in an abdominoplasty umbilicus finder device and method as shown and described in the following figures and related description.

### BRIEF DESCRIPTION OF THE DRAWINGS

Various embodiments of the present invention are shown and described in reference to the numbered drawings wherein:

FIG. 1 shows a perspective view of an umbilicus finder device;

FIG. 2A shows a perspective view of one aspect of the invention;

FIG. 2B shows a perspective view of yet another alternate construction of an umbilicus finder device;

FIG. 3A shows a top view of the umbilicus finder device of FIG. 1;

FIG. 3B shows a side view of the umbilicus finder device of FIG. 1;

FIG. 4A shows a side perspective view of one aspect of the invention wherein the device includes anchoring hooks;

FIG. 4B shows a top perspective view of the umbilicus finder device of FIG. 4B;

FIG. 5 shows a perspective view of another possible construction of the umbilicus finder device; and

FIG. 6 shows a side perspective view of an umbilicus finder device in place around the umbilicus during musculoaponeurotic plication in abdominoplasty.

It will be appreciated that the drawings are illustrative of various aspects and embodiments of the present invention and not limiting of the scope of the invention which is defined by the appended claims. The embodiments shown accomplish various aspects and objects of the invention, though it is not necessary that any embodiment or construction accomplish all aspects or any particular aspect of the invention. It is appreciated that it is not possible to clearly show each element and aspect of the invention. As such, multiple figures are presented to separately illustrate the various details of the invention in greater clarity. It will be understood that various structures shown in one figure may be used in a device shown in another figure.

### DETAILED DESCRIPTION

The invention and accompanying drawings will now be discussed in reference to the numerals provided therein so as to enable one skilled in the art to practice the present invention. The skilled artisan will understand, however, that the methods described below can be practiced without employing these specific details, or that they can be used for purposes other than those described herein. Indeed, they can be modified and can be used in conjunction with products and techniques known to those of skill in the art in light of the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment” or “an embodiment” means that a particular feature, structure, or characteristic described in connection with the embodiment is included in at least one embodiment. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the invention may be combined in any suitable manner in one or more embodiments. In the following description, numerous specific details are provided, such as examples of products or manufacturing techniques that may be used, to provide a thorough understanding of embodiments of the invention. One skilled in the relevant art will recognize, however, that embodiments of the invention may be practiced without one or more of the specific details, or with other methods, components, materials, and so forth. In other instances, well-known structures, materials, or operations are not shown or described in detail to avoid obscuring aspects of the invention.

Before the present invention is disclosed and described in detail, it should be understood that the present invention is not limited to any particular structures, process steps, or materials discussed or disclosed herein, but is extended to include equivalents thereof as would be recognized by those of ordinarily skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of the aspect in the claims.

It should also be noted that, as used in this specification and the appended claims, singular forms such as “a,” “an,” and “the” may include the plural unless the context clearly dictates otherwise. Thus, for example, reference to “a spring” may include one or more of such springs, and reference to “the layer” may include reference to one or more of such layers.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing the nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The Invention

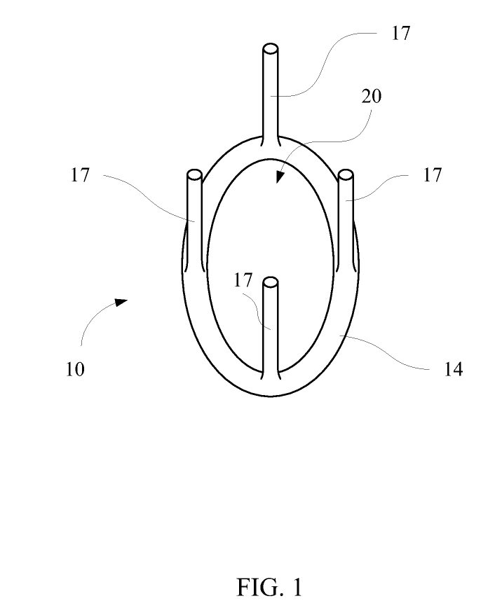

FIG. 1 shows a perspective view of an embodiment of the umbilicus finding device, indicated generally at 10. The device 10 is preferably generally oval-shaped, as typical umbilici are oval-shaped. The device 10 consists of a body or ring portion 14 and four projections or spikes 17 projecting upward. The projections or spikes 17 may extend, for example, generally 90 degrees out of the plane of the ring portion 14. The oval-shaped ring portion 14 defines a void 20, which is of a size suitable for fitting a typical umbilicus. As shown in FIG. 1, the projections or spikes 17 are spaced evenly along the ring and are located at the positions on the ring or body portion 14 corresponding to 0, 90, 180, and 270 degrees.

The four projections 17 may be of a length suitable to be easily felt through the closed subcutaneous fat layers, but not so long that they affect or change the closure of the incision. In one embodiment, the projections are about 15 millimeters in length. As shown in FIG. 1, the four projections are spaced generally equidistantly around the ring portion 14. However, such spacing is not required, and the projections may be placed at any position desired. Additionally, the projections 17 may be sharp enough to be felt easily through the layers of fat, but may or may not be so sharp as to cut through the dermis or epidermis. In a presently preferred embodiment, the projections are not sharp enough to cut through the dermis or epidermis, but are easily felt through the layers of fat.

The device may be constructed of any suitable material known in the art. For example, the device may be made of a plastic polymer, such as those currently known in the medical field. In one construction, the device may be made of injection-molded plastic. This plastic simplifies the manufacturing process, and can also be cut with heavy scissors or other tools if the surgeon desires to assist in removal of the device after its use.

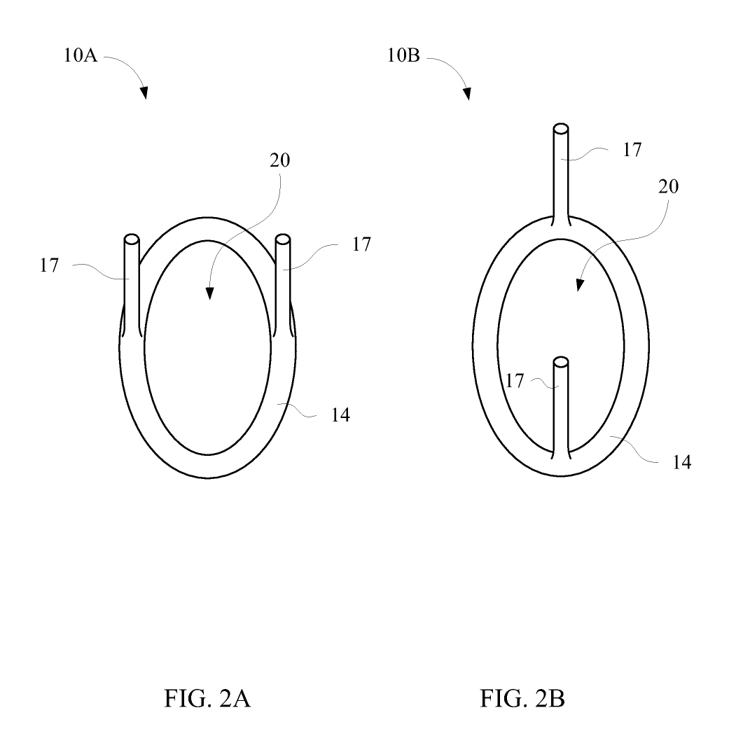

Turning now to FIGS. 2A and 2B, there are shown perspective views of alternate constructions of the umbilicus finder device, generally indicated at 10A and 10B, respectively. These alternate embodiments show umbilicus finder devices that use only two spikes or projections 17 extending from the body or ring portion 14. While an embodiment with four projections may be desirable, two projections may still allow proper location of the umbilicus. These may be, for example, located on opposite ends of the oval from one another (i.e., 180 degrees apart). As shown in FIG. 2A, the two projections 17 may be located at the 3 o'clock and 9 o'clock positions on the narrow diameter of the oval. Alternatively, FIG. 2B shows the two projections 17 at the 12 and 6 o'clock positions on the long diameter of the oval. One with skill in the art would appreciate that varying numbers of projections may be placed around the ring at varying positions and still achieve the desired effect of proper location of the umbilicus.

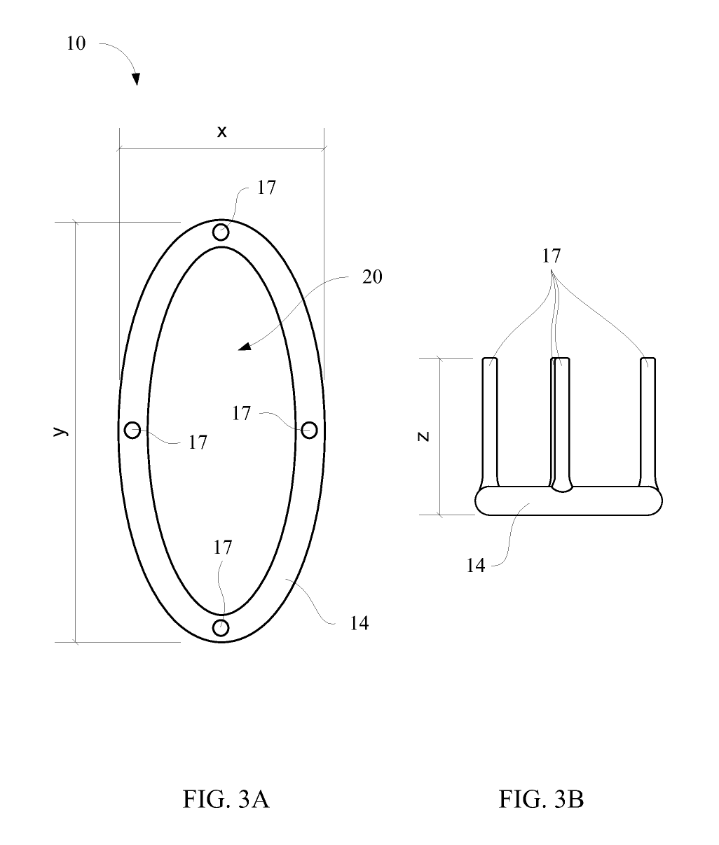

Turning now to FIGS. 3A and 3B, alternate views of the device according to FIG. 1 are shown, along with possible measurements for the size of the device. FIG. 3A shows a top-view of the umbilicus finder device. By way of example, the small diameter (x in FIG. 3A) of the oval or ring-portion 14 of the device 10 could be around 10-30 millimeters and the large diameter (y in FIG. 3A) could be around 30-50 millimeters, with 20×40 millimeters being common.

FIG. 3B shows a side-view of the device according to FIG. 1. Again, by way of example, the length of the projections 17 (z in FIG. 3B) could be about 10-25 millimeters, with 15 millimeters being common. A person having skill in the art will appreciate that these approximate sizes are around the size of a typical umbilicus, and that other sizes could be used depending on the patient.

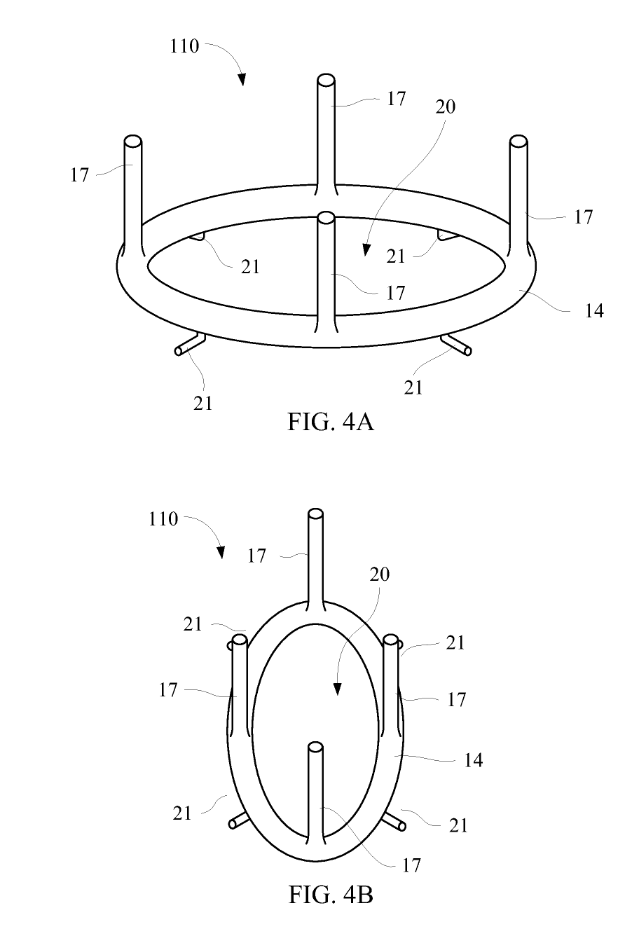

FIGS. 4A and 4B show yet another possible aspect of the umbilicus finder device, generally indicated at 110. FIG. 4A shows a side perspective view of the device 110 and FIG. 4B shows a top perspective view of the device. According to this aspect of the invention, one or more anchoring hooks 21 may be located at the bottom or under side of the ring portion 14 of the device 110. These anchoring hooks 21 may be used to help secure the device to the underlying fascia. Without the anchoring hooks, the surgeon may use, for example, tacking sutures to hold the device in place. The anchoring hooks 21 may allow be a simple method for the device 110 to be connected to the fascia.

According to this aspect, the device 110 may be constructed from injection-molded plastic or the like that is resilient. Thus, if the device is compressed, it may return to its original shape. This may allow the surgeon to press or squeeze the device 110, placing it around the umbilicus (the umbilicus being positioned in void or hollow 20), and press the device 110 into the surrounding fascia. The surgeon would then release the device 110, and the resiliency of the material the device is constructed from would allow it to return to its original position. As the device 110 returns to its original position, the anchoring hooks 21 may embed in the fascia, holding the device securely in the fascia of the abdominal wall around the umbilicus.

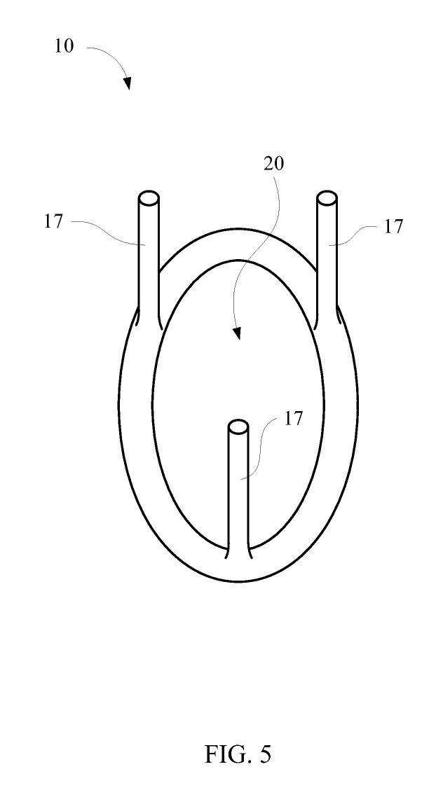

Additional embodiments of the current device could be used, and one having skill in the art would appreciate the numerous possibilities. For example, the device could have three projections evenly spaced around the generally-oval shaped ring. Thus, as shown in FIG. 5, the umbilicus finder device may have three projections. The device could also have a single projection, or multiple projections depending on the desire of the surgeon Likewise, while it is presently preferred to have the body or ring portion 14 form a complete ring or oval, it will be appreciated that the body may be of some other shape so that it may be attached to the umbilicus without forming a complete ring.

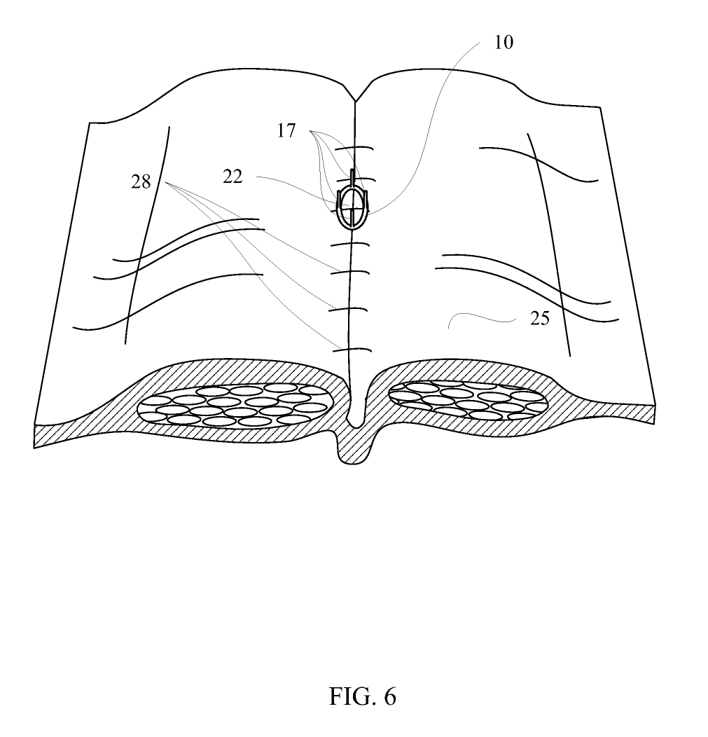

Turning now to FIG. 6, a side perspective view is shown of the device in place around the umbilicus during musculoaponeurotic plication (tightening of the musculofascial layer of the abdomen) in abdominoplasty. In use, the surgeon first makes an incision from hip to hip, elevating skin and fat layers from the abdominal wall fascia. Then a second incision is made to free the umbilicus (which is attached to the abdominal wall fascia tissue) from the surrounding skin. After lifting the abdominal flap, the surgeon may chose to plicate the musclofascial layer of the abdomen to tighten the abdominal wall. The surgeon then places the umbilicus finder device around the umbilicus. This may be accomplished, for example, by use of an umbilicus finder device 110 with anchoring hooks 21, or by using tacking sutures to tack the umbilicus finder device 10 into place around the umbilicus. The umbilicus finder device may be tacked into place around the umbilicus using, for example, 6-0 polypropylene or nylon suture or another suitable material known in the art. The device may also be held in place by any other suitable method.

In FIG. 6, the umbilicus finder device 10, with its projections 17, can be seen around the umbilicus 22. The device 10 separates the umbilicus 22 from the surrounding fascia 25 and enables the surgeon to feel the location of the umbilicus. This facilitates later cutting of a new orifice in the skin at the proper point of reattachment to the umbilicus.

Next, after placement of the umbilicus finder device, the surgeon proceeds with the abdominoplasty, stretching the flap of skin downward, removing the excess skin (including the previous orifice for the umbilicus) and suturing it into place. Once the flap is sutured in place, the surgeon locates the exact position of the umbilicus by feeling the projections or spikes 17 on the umbilicus finder device through the overlying skin and fat. These spikes may be sharp enough to poke through the layers of fat, but not the skin. The surgeon then cuts a new orifice at the location of the device, and removes the umbilicus finder device through the new orifice, either through an orifice large enough to remove the device whole, or, more commonly, by cutting the device to enable it to be pulled through a smaller orifice. The umbilicus is sutured into place, and the surgeon dresses the site.

There are numerous advantages to the present invention. For example, as the surgeon closes the various layers of skin, fat, and fascia, the exact location of the umbilicus relative to the overlying skin flap may shift. According to the present invention, the umbilicus finder device shifts with the umbilicus. Thus, the surgeon may close all layers of fat and epidermis and still readily determine the nearly exact, final position of the umbilicus.

Additionally, the device is of simple design and is easily deployed during surgery. The surgeon need only use tacking sutures or anchoring hooks provided on the device to attach the device to the fascia, and the projections or spikes allow for ready tactile determination of the exact location of the umbilicus without any further need for additional instruments or skill on the part of the surgeon. The device is easily removed through the same orifice created for the umbilicus.

It will be appreciated that the present invention can be expressed in multiple different configurations and methods. For example, in accordance with one aspect of the present invention an umbilicus finder device may include an oval-shaped body, the oval-shaped body defining a void, and at least one projection extending from the body. The device may also include a void which is sized to fit closely around a normal umbilicus of a human being. Furthermore, the device may have body having a plane and the at least one projection projecting upwardly 90 degrees from the plane of the body; the at least one projection being at least two projections; the at least two projections are evenly spaced around the body; the at least two projections being four projections; the projections being located at positions on the oval-shaped body corresponding to 0, 90, 180, and 270 degrees; the oval-shaped body has a short radius and a long radius; the short radius being between approximately 10 and 30 millimeters; the long radius being between approximately 30 and 50 millimeters; and/or the at least two projections being approximately 15 millimeters in length, and combinations thereof.

Likewise, the invention may include a device for locating an umbilicus, the device having an annular body, the annular body defining a hollow space for receiving an umbilicus, the annular body having at least one projection extending upwardly therefrom. Moreover, the device may have at least two projections extending from the annular body; the at least two projections being located at the superior and inferior points of the annular body, respectively; the annular body defining a plane with the at least one projection comprises first, second, third, and fourth projections projecting 90 degrees out of the plane of the body; the first, second, third, and fourth projections being spaced apart evenly on the annular body; and/or at least one anchoring hook extending downwardly from the annular body, and combinations thereof.

Furthermore, a method of the present invention may include attaching an umbilicus finder device to the fascia of the abdominal wall to or adjacent to the umbilicus, locating the location of the umbilicus by feeling the location of the umbilicus finder device through skin covering the abdomen, and forming a new umbilicus orifice. It may also include attaching the umbilicus finder device further comprises tacking the umbilicus finder device to the umbilicus; stretching abdominal skin over the umbilicus finder device; the umbilicus finder device having a plurality of projections and pressing on the skin to cause the projections to extend through fat layers under the skin to thereby provide a tactile location of the umbilicus; withdrawing the umbilicus finder device through the new umbilicus orifice; cutting the umbilicus finder device prior to withdrawal through the new umbilicus orifice; using at least a pair of tacking sutures attaching an umbilicus finder device to the fascia of the abdominal wall further comprising the step of attaching an umbilicus finder device to the fascia of the abdominal wall so as to encircle an umbilical stalk; and/or the umbilicus finder device further having at least one anchoring hook extending downwardly therefrom, and compressing the umbilicus finder and releasing the umbilicus finder such that the anchoring hooks embed in the fascia, and combinations thereof.

There is thus disclosed an umbilicus finder device and methods for using the same in locating the exact location of the new orifice for the umbilicus following abdominoplasty surgery. It will be appreciated that numerous changes may be made to the present invention without departing from the scope of the claims.

What is claimed is:

1. An umbilicus finder device comprising an oval-shaped body, the oval-shaped body defining a void, and at least one projection extending from the body.

2. The umbilicus finder device of claim 1, wherein the void is sized to fit closely around a normal umbilicus of a human being.

3. The umbilicus finder device of claim 2, wherein the body has a plane and wherein the at least one projection projects upwardly 90 degrees from the plane of the body.

4. The umbilicus finder device of claim 3, wherein the at least one projection comprises at least two projections.

5. The umbilicus finder device of claim 4, wherein the at least two projections are evenly spaced around the body.

6. The umbilicus finder device of claim 4, wherein the at least two projections comprise four projections, the four projections being located at positions on the oval-shaped body corresponding to 0, 90, 180, and 270 degrees.

7. The umbilicus finder device of claim 1, wherein the oval-shaped body has a short radius and a long radius.

8. The umbilicus finder device of claim 7, wherein the short radius is between approximately 10 and 30 millimeters.

9. The umbilicus finder device of claim 7, wherein the long radius is between approximately 30 and 50 millimeters.

10. The umbilicus finder device of claim 4, wherein the at least two projections are approximately 15 millimeters in length.

11. A device for locating an umbilicus, the device comprising an annular body, the annular body defining a hollow space for receiving an umbilicus, the annular body having at least one projection extending upwardly therefrom.

12. The device for locating an umbilicus of claim 11, wherein the device comprises at least two projections extending from the annular body.

13. The device for locating an umbilicus according to claim 12, wherein the at least two projections are located at the superior and inferior points of the annular body, respectively.

14. The device according to claim 11, wherein the annular body has a plane and wherein the at least one projection comprises first, second, third, and fourth projections; the first, second, third, and fourth projections projecting 90 degrees out of the plane of the body; the first, second, third, and fourth projections being spaced apart evenly on the annular body.

15. The device according to claim 11, wherein the device further comprises at least one anchoring hook extending downwardly from the annular body.

16. A method for locating an umbilicus during abdominoplasty surgery, the method comprising the steps of:
attaching an umbilicus finder device to the fascia of the abdominal wall to or adjacent to the umbilicus;
locating the location of the umbilicus by feeling the location of the umbilicus finder device through skin covering the abdomen; and
forming a new umbilicus orifice.

17. The method according to claim 16, wherein the step of attaching the umbilicus finder device further comprises tacking the umbilicus finder device to the umbilicus.

18. The method according to claim 16, wherein the method further comprises stretching abdominal skin over the umbilicus finder device.

19. The method according to claim 16, wherein the umbilicus finder device comprises a plurality of projections and wherein the method further comprises pressing on the skin to cause the projections to extend through fat layers under the skin to thereby provide a tactile location of the umbilicus.

20. The method according to claim 16, wherein the method further comprises withdrawing the umbilicus finder device through the new umbilicus orifice.

21. The method according to claim 20, wherein the method further comprises cutting the umbilicus finder device prior to withdrawal through the new umbilicus orifice.

22. The method of claim 16, the step of using at least a pair of tacking sutures attaching an umbilicus finder device to the fascia of the abdominal wall further comprising the step of attaching an umbilicus finder device to the fascia of the abdominal wall so as to encircle an umbilical stalk.

23. The method according to claim 19, wherein the umbilicus finder device further comprises at least one anchoring hook extending downwardly therefrom, and wherein the step of attaching the umbilicus finder device further comprises compressing the umbilicus finder and releasing the umbilicus finder such that the anchoring hooks embed in the fascia.
